(19) United States
(12) Patent Application Publication
(10) Pub. No.: US 2022/0151656 A1
(43) Pub. Date: May 19, 2022

(54) Intrathecal Needle Closed System and Method of Reducing Post Dural Puncture Headache and Infection

(71) Applicant: Blaine Cameron, Plantation, FL (US)

(72) Inventor: Blaine Cameron, Plantation, FL (US)

(21) Appl. No.: 17/529,176

(22) Filed: Nov. 17, 2021

Related U.S. Application Data
(63) Continuation of application No. 63/114,628, filed on Nov. 17, 2020.
(60) Provisional application No. 63/114,628, filed on Nov. 17, 2020.

(51) Int. Cl. A61B 17/34

(52) U.S. Cl. CPC A61B 17/3401; A61B 17/3498

(57) ABSTRACT

An intrathecal needle closed system for spinal needle that reduces the incidence of Post Dural Puncture Headache (PDPH) and infection. The intrathecal needle closed system generally includes closed system with CSF indicator, CSF indicator port, introducer needle, standardized needle size, and re-insertable stylet in closed system.

### CROSS-REFERENCE TO RELATED APPLICATIONS

This is a continuation application of Blaine Cameron, Intrathecal Needle Closed System and Method of Reducing Post Dural Puncture Headache and Infection, U.S. Appl. No. 63/114,628, filed Nov. 17, 2020 and incorporated herein by reference in its entirety.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable INCORPORATION-BY-REFERENCE OF MATERIAL SUBMITTED ON A COMPACT DISC

Not Applicable

### BACKGROUND OF THE INVENTION

### Field of the Invention

The risk of intrathecal procedure include post dural puncture headache (PDPH), infection. The 3 pathophysiologic mechanisms proposed are CSF hypotension with compensatory meningeal venodialation (vasodilation) and blood volume expansion, with headache caused by acute venous distention. Consistent with MRI findings.

Intracranial hypotension related to CSF leak which may cause sagging of intracranial structures and stretch of sensory intracranial nerves, causing pain and cranial nerve palsies. MRI findings of downward displacement of the brain.

Altered craniospinal elasticity post lumbar puncture resulting in increased caudal compliance relative to intracranial compliance and acute intracranial venodialation (vasodilation) in the upright position.

The incidence of PDPH may be up to 11% of cases.

Other complications of dural puncture Cranial nerve palsy, hearing loss, Hearing loss reported in up to 10-50% of patients though less than 25% aware of the deficit. Anesthesiology 2003:98:241, Anesth Analg 2002:95:198.

Pneumocephalus. Cranial nerve VI palsy. Subdural hematoma. Chronic low back pain, bacterial meningitis, and post-partum depression. Epidural blood patch is not indicated as treatment for any of these complications.

In rare cases Reversible cerebral vasoconstriction syndrome (RCVS) and posterior reversible encephalopathy syndrome (PRES).

### Description of the Related Art

### SUMMARY OF THE INVENTION

Example embodiments in general relate to an intrathecal needle closed system for spinal needle that reduces the incidence of Post Dural Puncture Headache (PDPH) and infection.

An example embodiment of the present invention is directed to an intrathecal needle (the “Cameron needle” or “Cameron Spinal Needle”), which includes closed system with CSF indicator, CSF indicator port, introducer needle, standardized needle size, and re-insertable stylet in closed system.

One object is to provide an intrathecal needle closed system for spinal needle that reduces the incidence of Post Dural Puncture Headache (PDPH) and infection.

Another object is to provide an intrathecal needle closed system that reduces the risk and incidence of Post Dural Puncture headache (PDPH).

Another object is to provide an intrathecal needle closed system that reduces the incidence of infection.

Another object is to provide an intrathecal needle closed system that confirms needle placement in the intrathecal space.

Another object is to provide an intrathecal needle closed system that reduces the incidence of failed spinal.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Features and advantages of the present invention will become apparent to those skilled in the art from the following description with reference to the drawings, in which:

Example embodiments will become more fully understood from the detailed description given herein below and the accompanying drawings, wherein like elements are represented by like reference characters, which are given by way of illustration only and thus are not limitative of the example embodiments herein.

FIG. 1: is a front view of the present invention

FIG. 2: is a side cross sectional view of the present invention

FIG. 3: is a zoomed side view of the needle tip of the present invention

FIG. 4: is a zoomed front view of the needle tip of the present invention

FIG. 5: is a view of a prior art intrathecal needle in use inserted into intrathecal space perpendicular to the direction of gravity

FIG. 6: is a view of the present invention in use inserted into intrathecal space perpendicular to the direction of gravity

FIG. 7: is a view of a prior art intrathecal needle in use inserted into intrathecal space perpendicular to the direction of gravity

FIG. 8: is a view of the present invention in use inserted into intrathecal space perpendicular to the direction of gravity

FIG. 9: is a tip down view of a prior art intrathecal needle in use inserted into intrathecal space

FIG. 10: is a tip down view of the present invention in use inserted into intrathecal space

### DETAILED DESCRIPTION OF THE INVENTION

### A. Overview

Turning now descriptively to the drawings, in which similar reference characters denote similar elements throughout the several views, the figures illustrate an example embodiment comprising closed system with CSF indicator, CSF indicator port, introducer needle, standardized needle size, and re-insertable stylet in closed system.

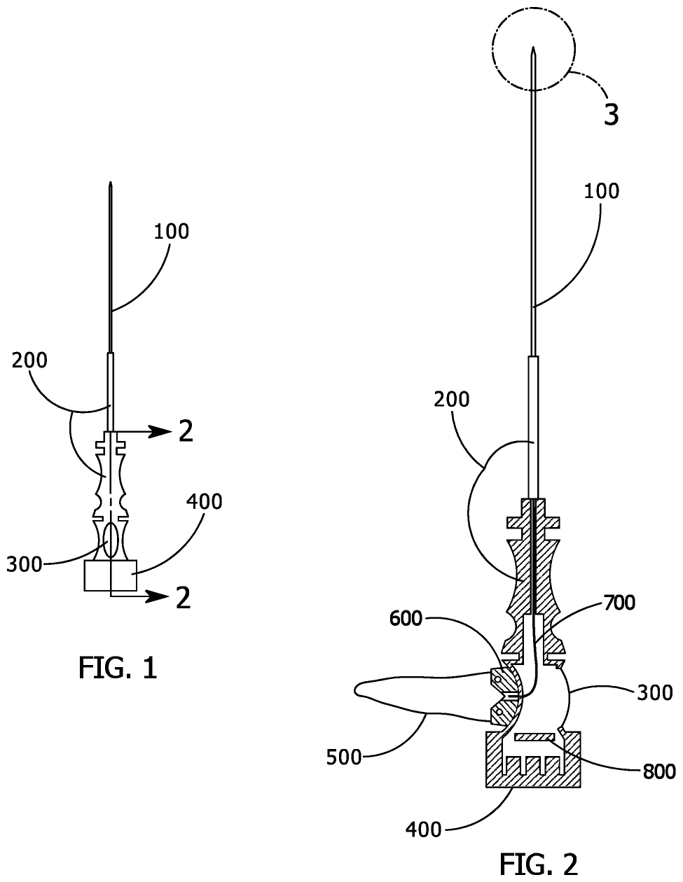

FIG. 1 shows: Cameron spinal needle 100; introducer needle 200; indicator side port 300; and, removable cap 400.

FIG. 2 shows: Cameron spinal needle 100; introducer needle 200; indicator side port 300; removable cap 400; stylet cover 500; stylet cap 600; malleable stylet 700; and, bidirectional valve 800.

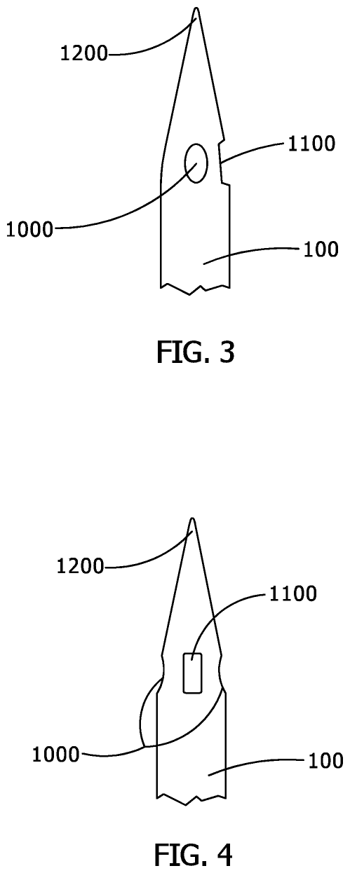

FIG. 3 shows: Cameron spinal needle 100; circular side port lateral 1000; square superior port 1100; and, pencil port 1200.

FIG. 4 shows: Cameron spinal needle 100; circular side port lateral 1000; square superior port 1100; and, pencil port 1200.

FIG. 5 shows: square superior port 1100; pencil port 1200; injectate direction 2000; intrathecal space 2100; direction of gravity 2200; and, Whitacre needle 2300.

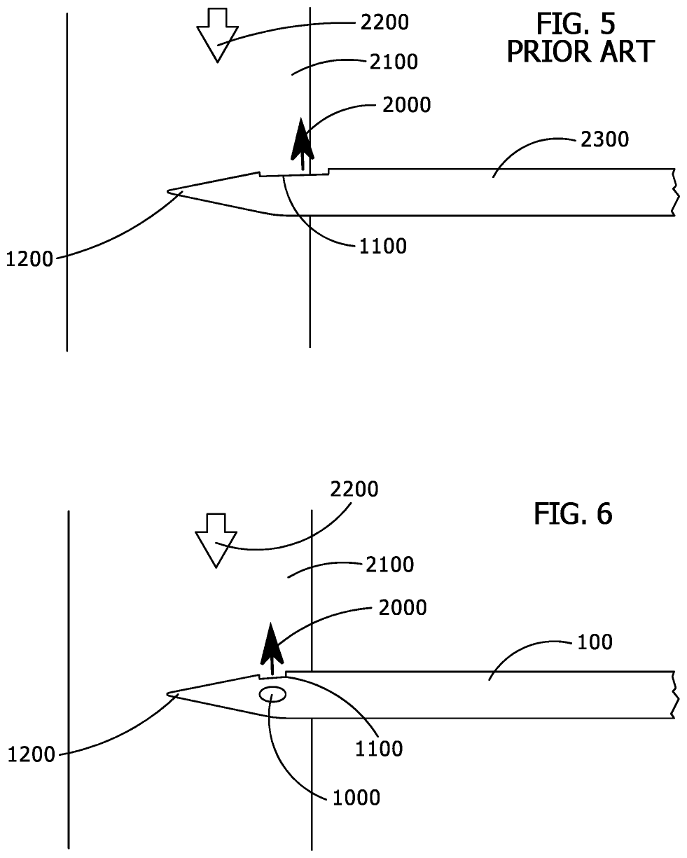

FIG. 6 shows: Cameron spinal needle 100; circular side port lateral 1000; square superior port 1100; pencil port 1200; injectate direction 2000; intrathecal space 2100; and, direction of gravity 2200.

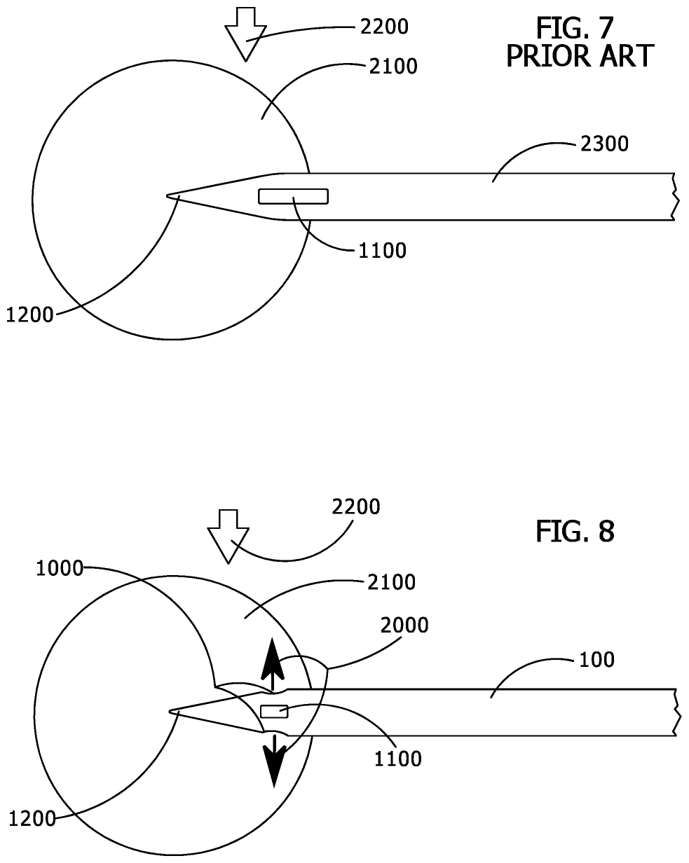

FIG. 7 shows: square superior port 1100; pencil port 1200; intrathecal space 2100; direction of gravity 2200; and, Whitacre needle 2300.

FIG. 8 shows: Cameron spinal needle 100; circular side port lateral 1000; square superior port 1100; pencil port 1200; injectate direction 2000; intrathecal space 2100; and, direction of gravity 2200.

FIG. 9 shows: square superior port 1100; pencil port 1200; injectate direction 2000; intrathecal space 2100; and, Whitacre needle 2300.

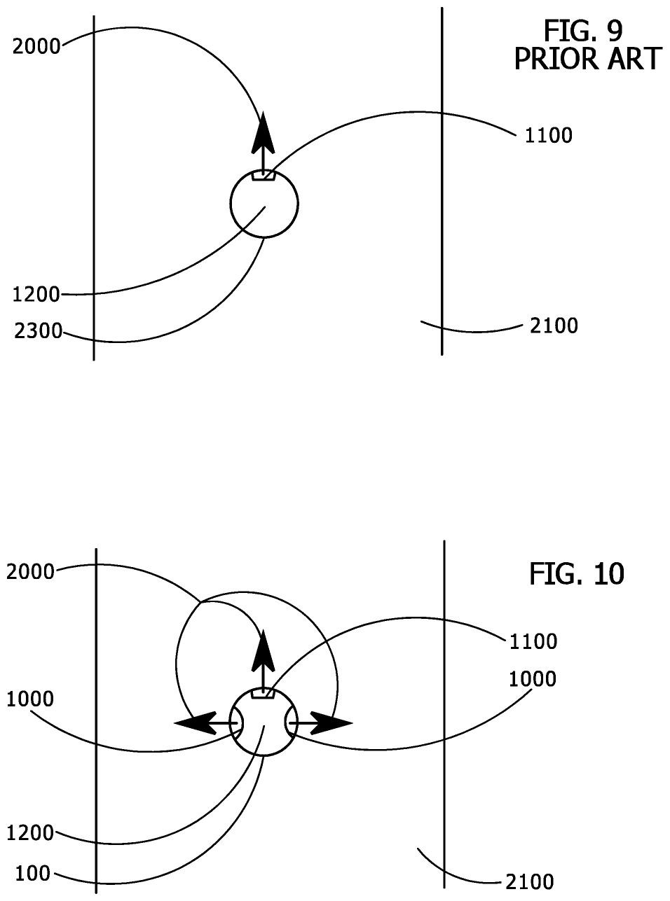

FIG. 10 shows: Cameron spinal needle 100; circular side port lateral 1000; square superior port 1100; pencil port 1200; injectate direction 2000; and, intrathecal space 2100.

### A. Cameron Needle Closed Distal Needle End

The distal end of the cameron needle 100 is a closed system with a valve which allows for injection or aspiration but does not allow for spontaneous egress of CSF fluid from the spine. By limiting the CSF exposure, the risk of contamination and therefore infection is reduced. The loss of CSF in intrathecal placement is reduced or potentially eliminated thus reducing the incidence of infection and PDPH.

The distal/provider end of the cameron needle 100 is free flowing up to the bidirectional valve, CSF is not passively lost but must be actively removed by aspiration if so desired. In the instance of intrathecal placement, no CSF is lost. Also, CSF is not exposed to the open and as such less likely to be contaminated and thus minimizing the risk of contamination and thus infection or intrathecal abscess or meningitis.

The stylet is malleable and bendable and is retracted superiorly and not at the traditional distal open end. The traditional open end has a removable cover or cap. This also reduces the surgeon/anesthesiologist/provider exposure to patient bodily fluids or CSF. This reduces provider exposure to potential exposure to HIV, Hepatitis C etc.

### B. Cameron Needle CSF Indicator

CSF indicator using Biuret, Piotrowski, Geiss reaction and others which can be located in a superiorly oriented position with needle opening in the translucent lumen or alternatively in a side port visualization chamber. The indicator can be used to identify protein, nitrite, glucose hemoglobin, pH to differentiate between CSF, and local anesthetic and plasma.

CSF indicator consists of a reagent treated substance incorporated into the superior translucent lumen and thus changes color when it encounters CSF thus confirming CSF and therefore intrathecal placement. The indicator differentiates between CSF and other fluids that are possible in the needle such as local anesthetic or plasma. The color change makes the presence of CSF more visible with a color change rather than the provider having to see clear CSF in a clear transparent needle distal end without having to lose CSF.

The reagent can consist of a novel reagent specific to the diagnosis of CSF or can consist of one of the many known reagents that can take advantage of the difference in the properties of CSF as opposed to local anesthetic or plasma. For example: protein; pH; glucose; heme; nitrate; potassium, etc.

### C. Cameron Needle Reinsertion Stylet in Closed System

The stylet can be removed to allow CSF but remain in a closed system thus allowing for free CSF flow to confirm correct placement in the intrathecal space while simultaneously allowing for reinsertion of the stylet which has not been removed from the closed system and thus reduce the incidence of PDPH without increasing the risk of infection. With a pencil point type needle, reinsertion of the stylet before removing the needle appears to lower the risk of PDPH. Absolute risk reduction 11%, 95% CL 6. 5-16. 2% J Neurol 1998:245:589.

Retractable stylet that is malleable. The stylet retracts superiorly into a sterile transparent attached bag. The stylet tip never leaves the stylet chamber fully due to a difference in length between the stylet and the bag. The stylet tip never leaving the needle facilitates the ease of reinsertion. The stylet never leaving the sterile transparent bag prevents the stylet leaving the closed system. The stylet never leaving the closed system prevents contamination of the stylet before reinsertion. Since the stylet never leaves the chamber this also prevents provider stylet stick injury.

The Cameron needle may have a hollow stylet with an open port to the indicator port. The stylet tip may be hard on both ends but malleable in the middle. Alternately the stylet tip may be fully retractable out of the lumen and needle chamber into an insertional side port located superiorly on the needle where the sterile bag attaches.

### D. Cameron Needle CSF Indicator Port

Side port in which CSF can enter before the valve that has the CSF indicator to confirm CSF and thus correct needle placement.

The port is on the inferior portion of the Cameron needle 100 but can be viewed through the transparent chamber. The indicator changes color based on the reagent used. The color change can indicate the presence of CSF, protein, glucose level, pH hemoglobin, nitrite etc. Some example, indicators include but are not limited to: Geiss; Piootrowski; Biuret; pseudoperoxidase; peroxide/tetramethylbenzidaine; tetrabromophenol blue; tetrachlorophenol; tetrabromosulplonphital; tetrahydrobenzopainoline bromomethynol blue; phenol red; etc.

### E. Cameron Needle CSF Valve

CSF valve that restricts the free flow of CSF beyond the valve. CSF can however be aspirated through the valve and alternatively medication such as local anesthetics and opioids can be injected through the valve. The valve is a two-way valve that does require minimal force for fluids to move through it, either injection or aspiration.

Bidirectional valve which stops passive flow but which allows active aspiration or injection without backward flow. Also is positioned in the chamber in such a way that it does not impact flow to the intraluminal indicator or the indicator side port.

Valve may be absent or removable in needles used to perform lumbar diagnostic punctures with measurement of opening pressures.

### F. Cameron Needle Pencil Point

The use of pencil point type spinal needles reduces the risk of PDPH compared to cutting needles of the same size. AnesthAnalg 200:91:916, Cochrane database syst rev 2017:4:CD010807, REG Anesth Pain Med 2018:43:502. The superior port is square and not rectangular, it is located near the tip of the needle. There is a unilateral or possible bilateral round port on the lateral side of the needle. This facilitates CSF entry and injectate entry into the CSF with less effect by patient position. Also, there is less potential CSF loss into the epidural space. There is less cephalad spread of injectate especially in the seated position.

Continuing the legacy of intrathecal anesthesia started by Leoard Corning 1885, 1891continued by Quincke, Bier's needle 1899Bainbridge 1900, Barker. 1898 Sicard cause of PDPH. 1922 Hoyt, Greene, needle 1926, 1950 Pitkin needle1 1927, Sise, Barke, Kirschner 1932, Rovenstine 1944, Hart and Whitacre, 1951 (Haroldson). The needle tip is closed, pencil point and small caliber. It minimizes CSF leak and damage to the dura and neural structures. It improves on the aforementioned.

Continuing the tradition of continuous spinal anesthesia in the tradition of Dean 1907, Lemmon 1940, malleable needle, 1943 Hingson annealed middle portion malleable. AN alternate of the needle tip is the Ballpen circa 2000. With the side port unilateral or bilateral. The system can be used for 34 gauge intrathecal catheter placement.

### G. Needle Orientation

A marker on the distal portion of the needle assists in orienting the needle in parallel as opposed to perpendicular/transverse. The superior square port close to the needle tip is oriented superiorly. Facilitating entry of CSF by gravity in a seated patient. The size and location and shape of the port minimizes the risk of CSF leak extra-durally. The oval side port facilitates the entry of CSF into the needle tip. The location of the lateral round ports facilitate entry of CSF into the needle especially during aspiration. The location of the ports facilitate entry of injectate into the intrathecal space with little barbotage. The lateral ports also facilitate CSF entry into the needle with the patient in the lateral position while the superior port is in the seated position. The lateral ports also minimize cephalad spread of injectate into the intrathecal space.

The needle is oriented with the superior needle tip orifice oriented in the cephalad direction, the indicator port is oriented in an inferior direction. The indicator intraluminally is in the superior orientation. The stylet is also oriented in such a way as it is retracted superiorly.

The needle orientation can be eliminated as one can perceive the needle orientation based on the position of the stylet provider end.

### H. Needle Size

Needle size is standardized to 27 gauge. For more complex procedural placement there is a shorter larger gauge 18 gauge insertion needle through which the needle can be inserted. This insertion needle helps to puncture the skin thus reducing needle contact with skin and thus skin flora thus reducing the incidence of infection. This technique also reduces damage to the needle tip.

Needle size is 27 gauge with an 18 gauge introducer needle.

Needles can be made in other sizes, but the preferred size of the intrathecal portion is 27 gauge. With the needle advanced with the stylet in place. The stylet can be hollow with an opening port at the inferior portion of the stylet in proximity with the indicator port with the stylet fully inserted. This gives a quicker flash of CSF allowing for quicker needle placement confirmation with a reduced loss of CSF. This alternate stylet completely occludes the ports except for one of the bilateral side ports thus preventing tissue occlusion in the remaining 2 needle tip ports.

### I. Bidirectional Valve

The bidirectional valve is located proximal to the provider but distal to the patient and near the provider end of the needle. It is beyond the indicator and the indicator port and thus does not interfere with free flow of CSF to these areas. In a diagnostic lumbar puncture with opening pressures is desired a version of the needle without the bidirectional valve would be needed. However, if CSF sample is needed only the bidirectional valve reduces contamination and excess loss of CSF. This limits the possible contamination of the CSF and thus makes viral and bacterial testing of CSF more accurate but reducing contamination of the sample.

Bidirectional valve is contained in the lumen and near the end cap but after the indicator side port and the exit for the re-insertable stylet. This portion may be removeable or alternatively absent for needles used in diagnostic lumbar punctures used to evaluate opening CSF pressures.

### J. Connections of Main Elements and Sub-Elements of Invention

The sub elements are all contained within the closed system. The removeable cap is at the end of the system and converts the system from a closed system on the provider side to an open one.

### K. Alternative Embodiments of Invention

Cameron needle can have the bidirectional valve or have the valve absent or removeable.

### L. Operation of Preferred Embodiment

Most improvements to the intrathecal needle have taken place at the needle tip. The needle has incorporated CSF indication and other substance indication at the provider. The stylet is re-insertable in a closed system. There is an indicator port allowing for faster recognition of placement in the intrathecal space with confirmation via indicator color change.

The bidirectional valve limits passive CSF loss. While at the needle tip the pencil point design is used but also incorporated is a 3 port which allows for improved CSF flow and smaller superior port to improve structural integrity with stylet in place during insertion.

Unless otherwise defined, all technical and scientific terms used herein have the same meaning as commonly understood by one of ordinary skill in the art to which this invention belongs. Although methods and materials similar to or equivalent to those described herein can be used in the practice or testing of the intrathecal needle closed system suitable methods and materials are described above. All publications, patent applications, patents, and other references mentioned herein are incorporated by reference in their entirety to the extent allowed by applicable law and regulations. The implant electrode system may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Having thus described preferred embodiments, it should be apparent to those skilled in the art that certain advantages of the described system have been achieved. It should also be appreciated that various modifications, adaptions, and alternative embodiments thereof may be made within the scope and spirit of the present invention. The invention is further defined by the following claims.

What is claimed is:

1. An Intrathecal Needle Closed System comprising:
at least one introducer needle;
at least one indicator side port;
at least one removable cap;
at least one stylet cover;
at least one stylet cap;
at least one malleable stylet;
at least one bidirectional valve;
at least one circular side port lateral;
at least one square superior port; and,
at least one pencil point.

2. A method for reducing incidence of infection by using an Intrathecal Needle Closed System comprising:
at least one introducer needle;
at least one indicator side port;
at least one removable cap;
at least one stylet cover;
at least one stylet cap;
at least one malleable stylet;
at least one bidirectional valve;
at least one circular side port lateral;
at least one square superior port; and,
at least one pencil point.

3. A method for reducing incidence of Post Dural Puncture Headache using an Intrathecal Needle Closed System comprising:
at least one introducer needle;
at least one indicator side port;
at least one removable cap;
at least one stylet cover;
at least one stylet cap;
at least one malleable stylet;
at least one bidirectional valve;
at least one circular side port lateral;
at least one square superior port; and,
at least one pencil point.
